(19) United States
(12) Patent Application Publication
(10) Pub. No.: US 2002/0018780 A1
(43) Pub. Date: Feb. 14, 2002

(54) Epitope-based vaccine for respiratory syncytial virus F-protein

(76) Inventors: Scott Koenig, Rockville, MD (US); Mark S. Hanson, Clarksville, MD (US); JoAnn Suzich, Washington Grove, MD (US); Nancy Ulbrandt, Gaithersburg, MD (US)

Correspondence Address: CARELLA, BYRNE, BAIN, GILFILLAN,, CECCHI, STEWART & OLSTEIN, 6 Becker Farm Road, Roseland, NJ, 07068, US

(21) Appl. No.: 09/865,499

(22) Filed: May 25, 2001

Related U.S. Application Data
(60) Provisional application No. 60/206,946, filed on May 25, 2000.

(51) Int. Cl. A61K 39/12; A61K 39/42

(52) U.S. Cl. 424/186.1; 424/147.1

(57) ABSTRACT

The present invention relates to a vaccine that comprises selected epitopes within the F protein structure that have been proven to specifically interact with known potent neutralizing antibodies while simultaneously being presented as part of a synthetic structure that offers these epitopes apart from the other non-neutralizing antigenic determinants of the virus but held in a native conformational form and thereby capable of eliciting neutralizing antibodies.

This application claims priority of U.S. Provisional Application No. 60/206,946, filed May 25, 2000, the disclosure of which is hereby incorporated by reference in its entirety.

### FIELD OF THE INVENTION

The present invention relates to vaccines useful against respiratory disease, especially respiratory infections caused by respiratory syncytial virus (RSV), wherein such vaccines employ specific epitopes within selected proteins present on the surface of viruses for producing a highly selected and potent antibody with lowered toxicity and other non-specific immunological reactions.

### BACKGROUND OF THE INVENTION

Respiratory syncytial virus (RSV) is a negative strand RNA Paramyxovirus of the Pneumovirus genus which often infects the upper and lower respiratory tract and is a major cause of contagious respiratory infection in children. Most infections are localized to the upper respiratory tract with symptoms no worse than a common cold although infants, the elderly and people with certain cardiovascular conditions may show more severe symptoms, with the infection often spreading to the lower respiratory tract. Some of these cases may even prove fatal. Respiratory syncytial virus (RSV) is a leading cause of serious lower respiratory tract disease in children and has been increasingly recognized as an important pathogen in the elderly.

Various antigenic sub-groups of RSV have been observed, based mostly on the amino acid sequence of the surface attachment G protein, the latter often showing wide variation in sequence from one isolate to another. [See: Johnson et al, Proc. Natl. Acad. Sci., 84, 5625-5629 (1987)] Conversely, the F (for fusion) protein, is a fairly well conserved polypeptide of approximately 70 kDa. [See: Johnson et al, J. Gen. Virol. 69, 2623-2628 (1988) and Johnson et al, J. Virol., 10, 3163-3166 (1987)].

Studies of the structure of the F-protein show it to be a homodimer formed from two single chain linear precursors, containing disulfide-linked fragments of about 48 and 23 kD, respectively. This latter 23 kD fragment has been found to be a prime target for antibodies capable of inhibiting formation of syncytia. The 48 kD, not the 23 kD, is the prime target for antibodies.

Strategies for prevention of RSV have included attempts at vaccination as well as passive administration of polyclonal and highly specific monoclonal antibodies against the F protein. Thus far, RSV vaccines have failed to surpass the immune response to the natural infection and induce the production of high levels of the neutralizing antibodies necessary to control the virus.

Heretofore, vaccination strategies against RSV have included use of inactivated virus, live attenuated virus, and subunit vaccines, with many of the latter employing F protein as an immune target. These have uniformly met with, at best, limited success.

Parenterally administered vaccines comprising RSV proteins, or subunits thereof, have exhibited modest immunological potency with respect to eliciting neutralizing antibodies. A subunit vaccine candidate for RSV consisting of F glycoprotein from RSV infected cell cultures and then purified by immunoaffinity chromatography has been described (See: Crowe, Vaccine, 13, 415-421 (1995)]. Parenteral immunization of seronegative or seropositive chimpanzees with this preparation, upon subsequent challenge with wild-type RSV, showed no effect on virus shedding or in the upper respiratory tract. In some rodents, the immune response to immunization with RSV F protein resulted in disease enhancement.

F protein has been purified from RSV-infected cells (Murphy et al, Vaccine, 7, 533 (1989); Hancock et al, Vaccine, 13, 391-400 (1995)) and has been prepared recombinantly using baculovirus (Wathen et al, J. Infect Dis., 163, 477-482 (1991)). In animal models of RSV infectivity, such as the cotton rat model, subunit vaccines were found to significantly reduce the viral load in the lungs of animals following RSV challenge. However, in some cases, enhanced pulmonary pathology was observed in animals that received the subunit vaccines and were subsequently challenged with RSV. In addition, it has been reported that while the F protein based subunit vaccines induced a potent anti-F response measured by ELISA, the level of RSV neutralizing antibodies that were generated was relatively low. In fact, one recent study (Sakurai et al, J. Virol. 73, 2956-2962 (1999)) reported evidence that antibodies generated during human RSV infections that interact with an immunodominant epitope of the F protein have high affinity for purified F protein but low affinity for F protein on RSV virions and lack virus-neutralization activity leading to the conclusion that F protein subunit vaccines will be at a disadvantage for maintaining the F protein neutralizing epitope(s) of the intact virion.

In sum, although use of strategies like subunit vaccines have succeeded in producing responsive antibodies, such antibodies were substantially non-neutralizing in character and thus provided inadequate protection against RSV.

In conclusion, a protective response against RSV is contingent on the production of neutralizing antibodies against the major viral surface glycoproteins while minimizing non-protective or pathological immune responses. The present invention avoids such problems by providing a vaccine that comprises selected epitopes within the F protein structure that have been shown to specifically interact with known potent neutralizing antibodies. These epitopes are presented as part of a synthetic structure that offers these epitopes apart from the other non-neutralizing antigenic determinants of the virus but and holds them in a native conformational form and thereby elicits neutralizing antibodies.

### BRIEF SUMMARY OF THE INVENTION

The present invention relates to vaccines comprising polypeptide structures that comprise selected epitopes within the F protein structure as part of a non-RSV polypeptide “framework” or “scaffold” wherein the selected epitopes are held in a conformation eliciting neutralizing antibodies against RSV.

It is therefore an object of the present invention to provide an immunogen for eliciting neutralizing antibodies against respiratory syncytial virus (RSV), said immunogen comprising a non-RSV polypeptide segment fused to a neutralizing epitope derived from the F protein of RSV, said immunogen being free of (including depleted of) immunodominant neutralizing RSV epitopes. Thus, said immunogen may be free of such non-neutralizing epitopes because such sequences have been prepared, or synthesized, without the segments corresponding to these non-neutralizing epitopes or, where the sequence is taken directly from a naturally occurring specimen, such specimen has been depleted of such non-neutralizing sequences.

In specific embodiments of the present invention, such immunogen comprises one or more polypeptides.

In other embodiments, said RSV neutralizing epitope is derived from the A/II region of the F protein including where the protein is the amino acid sequence of the A/II region of the F protein (i.e., the naturally occurring, or wild type, sequence). As used herein, the term “derived” includes sequences similar but not identical to the sequence of the epitopes disclosed herein as well as fragments of sequences otherwise identical to the sequences of said epitopes.

It is another object of the present invention to provide an immunogen as described herein where the conformational epitope that is inserted into the non-RSV polypeptide segment, or fragment, or portion, is an epitope that is recognized by the humanized antibody whose amino acid sequence is disclosed in FIGS. 7 and 8 of U.S. Pat. No. 5,824,307, including the modified humanized recombinant antibody referred to herein as MEDI-493, described below.

While it is to be understood that the preferred RSV epitope is defined as an epitope that binds to MEDI-493, it is also to be understood that such epitope may bind to antibodies other than MEDI-493.

It is an additional object of the present invention to provide a framework structure for holding the RSV F protein-derived polypeptide in a conformationally advantageous structure (i.e., one that elicits neutralizing antibodies). In specific embodiments, the recited non-RSV fragment and said F protein-derived polypeptide that defines an RSV epitope are joined to each other by covalent bonds, which are commonly peptide bonds.

It is a further object of the present invention to provide an immunogenic composition comprising at least one of the immunogens as disclosed herein wherein said immunogen is suspended in a pharmacologically acceptable carrier and vaccines, or vaccine compositions, comprising said immunogens and immunogenic compositions.

It is a still further object of the present invention to provide a process for preventing or treating a disease comprising administering to a patient having said disease, or at risk of contracting said disease, a therapeutically, or prophylactically, effective amount of the vaccine composition hereinabove described. In specific embodiments, such disease is a disease of the respiratory system, especially where said disease is caused by a virus, and most especially where said virus is respiratory syncytial virus (RSV).

### BRIEF DESCRIPTION OF THE DRAWINGS

FIG. 1 is a schematic ribbon diagram showing the overall conformation of EETI-2 protein from squash with its cystine-stabilized α-sheet motif (with its 3 β-strands bounded by loops and an α-helix). Below the structure is the amino acid sequence (SEQ ID NO: 1 and using standard one-letter codes) with the location of the disulfide bonds indicated by the lines.

FIG. 2 is a diagram showing a structure for EETI-2 protein and indicating the point of insertion of an epitope such as that described herein. The corresponding amino acid sequence is shown below with the location of a potential insert indicated thereon.

FIG. 3 is a diagram showing a structure for EETI-2 protein but indicating a point of insertion of an epitope or polypeptide insert different from that of FIG. 2. The corresponding amino acid sequence is below with the location of a potential insert indicated.

FIG. 4 shows sample scaffold inserts. Here, SK1 is the scaffold with the site of insertion at amino acid 3 and the SK2 is the scaffold with the site of insertion at amino acid number 18. The insertions are either 0 amino acids, 20 amino acids from the F protein corresponding to amino acids 255-275, or 63 amino acids corresponding to amino acids 218-281 of the F protein. Induction of product expression was carried out by the addition of IPTG. The left portion of the figure is the SDS PAGE gel stained for protein with SyproRed and the right side of the figure is a western blot probed with Medi493 and visualized by the activity of a secondary antibody against human IgG conjugated to alkaline phosphatase (as per the protocol described in Example 1).

FIG. 5 shows nucleotide sequences for Squash Knot I and II (SEQ ID NO: 2 and 4, respectively) and amino acid sequences SEQ ID NO: 3 and 5, respectively) with indicated restriction sites.

FIG. 6 shows nucleotide sequences for RSV Fusion-Protein. Amino acid sequence (255-275, SEQ ID NO: 6; and 218-281, SEQ ID NO: 8) with corresponding nucleotide sequences (SEQ ID NO: 7 and 9, respectively) with indicated restriction sites.

### DETAILED DESCRIPTION OF THE INVENTION

The present invention solves the problems of previously evaluated vaccine candidates by providing highly immunogenic recombinant polypeptides containing specific virus-neutralizing epitopes, or epitopic domains, or antigenic domains, or antigenic determinants, present in the fusion, or F, protein of respiratory syncytial virus (RSV) which, when used as an immunogen, provide a means of eliciting the production of highly specific neutralizing antibodies uniquely suitable for providing protection against RSV infection or for ameliorating an already existing infection.

More specifically, the present invention relates to vaccine comprising polypeptide structures that comprises selected epitopes within the F protein structure that have been proven to specifically interact with known potent neutralizing antibodies while simultaneously being presented as part of a non-RSV structure wherein the epitope has a conformational form that elicits neutralizing antibodies.

In accordance with a preferred embodiment of the present invention, the immunogen uses neutralizing epitopes resident on the mature F protein found on infectious virions without the inclusion of non-neutralizing epitopes.

In a preferred embodiment, the immunogen does not include RSV epitopes that recognize or produce non-neutralizing antibodies whereby the immunogen contains only neutralizing epitopes.

An immunogen according to the present invention comprises one or more neutralizing epitopic domains within the F protein and avoids use of domains not required for neutralizing activity. The determination of which domains are useful for the immunogens of the present invention is made based on the ability of specific epitopes, or epitopic domains, within the structure of the viral F protein to interact specifically and strongly with antibodies known to have strong neutralizing activity. Such antibodies include, for example, any of the RSV-neutralizing antibodies disclosed in FIGS. 7 and 8 of Johnson, U.S. Pat. No. 5,824,307. The antibodies described for use in the present invention have been shown to interact strongly with specific epitopes on the F protein of RSV and said antibodies are recombinant in nature. (see: Johnson et al, J. Infect Dis., 176, 1215-1224 (1997) describing MEDI-493, a recombinant antibody with good RSV neutralizing ability). It should be noted that, not all antibodies specific for RSV are necessarily neutralizing in character. (See: Johnson et al, J. Infect. Dis., 180, 35-40 (1999)) Thus, the present invention provides a means of eliminating non-neutralizing, potentially immunodominant epitopes from vaccine candidates.

Neutralizing monoclonal antibodies against human RSV F protein have been developed and have been shown capable of neutralization (i.e., preventing infection) of otherwise susceptible cultured mammalian cells. Competitive binding immunoassays with panels of such monoclonal antibodies have revealed that they bind primarily to three (Beeler and van Wyke Coelingh, J. Virol., 63, 2941-2950 (1989)) or more (Arbiza et al, J. General Virology, 73, 2225-2234 (1992)) non-overlapping antigenic sites on the F protein. Epitopes within antigenic site A, also called site II, are among the neutralizing sites most conserved among various strains of the two subtypes A and B of RSV. The mouse monoclonal antibody 1129 studied by Beeler was one of 6 monoclonal antibodies specific for antigenic site A/II and found to neutralize 13 of 14 clinical RSV isolates (Beeler and van Wyke Coelingh, 1989, above). Nucleotides encoding the combinatorial determining region sequences of monoclonal antibody 1129 were fused to human antibody framework regions to create a humanized monoclonal antibody designated MEDI-493 (Johnson et al, 1997, above). MEDI-493 retains the binding specificity of mouse 1129 and is highly potent for neutralization of RSV strains of diverse origin and protective in the cotton rat model. MEDI-493 is also the humanized antibody of U.S. Pat. No. 5,824,307, the disclosure of which is hereby incorporated by reference in its entirety. This humanized antibody is also effective in preventing RSV disease when administered to at-risk infants.

The epitopes of antigenic A/II appear to be localized to the amino terminal third of the F₁ fragment of the F protein. Under selection with A/II site (the A site or site II, whichever it is called) monoclonal antibodies, RSVs with single mutations in amino acids N²¹⁶, L²⁵⁸, N²⁶², N²⁶⁸, K²⁷², or S²⁷⁵ were able to escape neutralization in vitro (Arbiza et al, 1992; Lopez et al, 1998). Thus, amino acids spanning 60 residues influence the structure of the A/II site epitope(s). Synthetic peptides spanning residues 250-273, 255-275, and 258-271 failed to bind the neutralizing A/II site monoclonal antibodies (MAbs) in one such study (Arbiza et al, 1992), except for one MAb that bound peptide 255-275 but not closely related peptides. In a separate study, the ability of A/II site MAbs to bind synthetic, unconstrained peptides derived from the F protein sequence was related to peptide length. Thus, a greater number of A/II site MAbs bound a 61 amino acid residue peptide encompassing F protein amino acids 215-275 than reacted with a shorter peptide composed of 41 F protein amino acids 235-275. More A/II site MAbs reacted with this 41-mer than recognized a 21-residue peptide composed of F protein amino acids 255-275. Increased antigenicity correlated with a more ordered (less random) conformation in solution of the larger peptides Lopez et al, J. Gen. Virol., 74, 2567-2577 (1993)). These and other observations indicate that the A/II site MAbs, such as the humanized 1129 already referred to, recognize an as yet undefined three-dimensional conformation on the F protein that may include amino acids within the 216-275 sequence. The secondary structure of the F protein near this sequence was predicted by computer modeling to form a helix-loop-helix (Lopez et al, 1998).

However, while F protein-derived peptides as long as 61 amino acids can react with site A/II neutralizing MAbs, these peptides are very poor at eliciting neutralizing antibodies when used to immunize mice, even with the powerful Freund's complete adjuvant (Lopez et al (1993)).

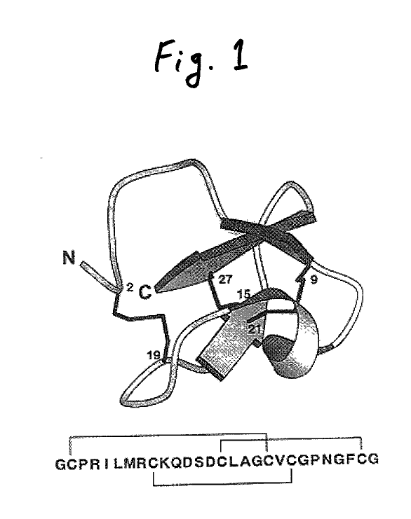

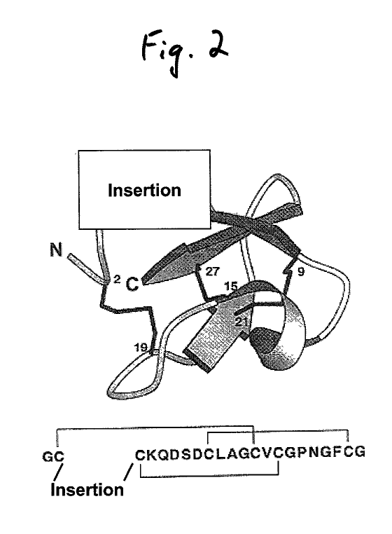

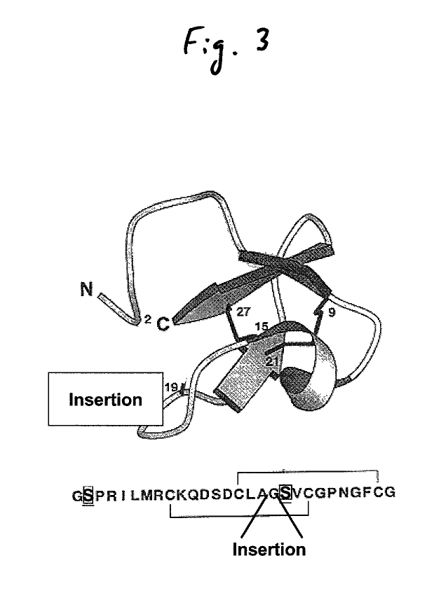

In accordance with the present invention, segments of varying length comprising all or part of the RSV F protein, especially the A/II site of the F protein, and most especially sequences identical to all or part of this sequence, are readily expressed as recombinant insertions into a heterologous protein “scaffold” or “framework” to provide an immunogen of the present invention. Alternatively, such immunogens may be synthesized directly. In accordance with the present invention, the “scaffold” protein, or “framework” protein, is chosen so as to include structural motifs, such as loop structures, such as those in the protein shown in FIG. 1, which can be replaced in whole (FIG. 2) or in part (FIG. 3) to result in a structure that holds the epitope in a conformation for eliciting neutralizing antibodies but absent the rest of the virus.

In accordance with the methods disclosed herein, by expressing portions of the F protein in Escherichia coil the minimal epitope capable of binding a potent neutralizing antibody, such as MEDI-493 (recited above), is readily determined.

In a specific embodiment, such an epitope comprises the amino acid fragment contained within amino acids 218 to 281 of the F protein from site A/II (strain long) described in Example 1. Of course, the immunogens disclosed herein are in no way limited to the exact amino acid sequences of the F protein, or even of the A/II site therein. Using techniques well known to those of skill in the art, and which will not be described further herein, epitopic sequences similar to, but not identical to, those found in such neutralizing epitopic sites of the F protein (as disclosed herein as examples only) are readily generated and, using the methods of the present invention, tested for their ability to react with neutralizing antibodies. In this way, the ability of the immunogens of the present invention to elicit neutralizing antibodies can be enhanced. It is also well known that the identity of the amino acids flanking such epitopes (such as the scaffold structures herein) can influence antigenicity. [See: Leclerc et al, Int Rev. Immunol., 11, 123-132 (1994); Zhang et al, Biol. Chem., 380, 365-374 (1999)).

Further, in accordance with the present invention, experimental animals, such as mice, are immunized with such enhanced-immunogens and the resulting antiserum tested for neutralization of RSV. Chimeric epitope subunit vaccines eliciting neutralizing titers higher than those following RSV infection are then evaluated for efficacy against RSV challenge.

In accordance with the foregoing, the present invention relates to an immunogen for eliciting neutralizing antibodies against respiratory syncytial virus (RSV), said immunogen comprising a non-RSV polypeptide fragment, wherein said fragment comprises a structural motif having at least one loop region that includes a polypeptide insert and wherein said polypeptide insert comprises a conformational epitope derived from the site A/II region of the F protein of RSV.

The immunogen of the present invention may be in the form of a single chain polypeptide or may be comprised of more than one chain with the individual chains linked together. Similarly, more than one epitopic structure may be part of the same immunogenic structure.

The immunogens of the present invention includes a scaffold or framework for holding the neutralizing epitopic structure in a conformationally correct structure for presentation to the immune system. Such a scaffold or framework commonly contains at least a segment, such as a loop structure shown in the polypeptide of FIG. 1, into which is inserted a segment, or fragment, or portion of an epitopic sequence derived from RSV F protein, commonly in the form of a polypeptide insert. However, the present invention contemplates embodiments where more than one such loop region is present in the same framework or scaffold structure.

In specific embodiments, the present invention includes immunogens wherein the scaffold or framework comprises more than one polypeptide insert, each present as part of a separate loop structure. The present invention further relates to immunogens in which the polypeptide insert comprises at least one conformational epitope derived from the site A/II region of the F protein of RSV. Such epitopes may include overlapping regions within the A/II region of the F protein and may also include more or less than the sequence of the A/II region.

The present invention also provides for a means of locating neutralizing epitopes within the F protein of RSV (or elsewhere, including other viruses) and, more importantly, for determining that such epitopes are present in a conformation that generates neutralizing antibodies. It is essential for eliciting neutralizing antibodies that the immune system be presented with F protein epitopes present in their native conformation, but without the presence of otherwise immunodominant “decoy” epitopes that elicit large amounts of antibody from the immune system of an infected individual but little of which represents neutralizing antibodies, thereby causing the immune system of an infected individual to waste resources on making non-neutralizing (and useless) antibodies while the virus escapes destruction.

Thus, in accordance with the present invention, epitopes useful in forming immunogens that elicit neutralizing antibodies are structures that conformationally mimic the native F protein but absent the rest of the virus. In place of the virus is the non-RSV fragment or polypeptide that acts as a scaffold or framework to hold the epitopic structure in this active conformation which is capable of eliciting neutralizing antibodies. The fact that the conformation of the epitope is correct is demonstrated by further testing of the scaffolded, or frameworked, epitope with potent neutralizing antibodies to show binding. Thus, the potent neutralizing antibodies described herein are used both to identify the desired epitopes as well as to ensure conformational stability following formation of the scaffolded, or frameworked, structure comprising the inserted polypeptide or epitope. Thus, the present invention provides a means finding only those epitopes that elicit neutralizing antibodies as well as ensuring the conformational integrity of such epitopes following insertion into the scaffold or framework structures disclosed herein.

In a specific embodiment of the immunogens of the present invention, the scaffold or framework structure is derived from the sequence of EETI-2 protein as shown in FIGS. 1, 2, and 3.

As used herein, the terms “portion,” “segment,” and “fragment,” when used in relation to polypeptides, refer to a continuous sequence of residues, such as amino acid residues, which sequence forms a subset of a larger sequence. For example, if a polypeptide were subjected to treatment with any of the common endopeptidases, such as trypsin or chymotrypsin, the oligopeptides resulting from such treatment would represent portions, segments or fragments of the starting polypeptide.

In one embodiment of the present invention, the neutralizing RSV epitope (or epitope that reacts specifically with neutralizing antibodies) is joined to the remainder of the scaffold or framework by covalent bonds, commonly peptide bonds. However, other means of joining these structures may be contemplated within the bounds of the invention disclosed herein.

In one embodiment, the present invention relates to a polypeptide insert having the amino acid sequence of the figures and, once scaffolded, forming the structures based on FIGS. 2 and 3 (with sequences as shown therein, respectively).

The polypeptide insert of the present invention may be a recombinant polypeptide, a natural polypeptide or a wholly synthetic polypeptide.

The polypeptide insert present in an immunogen of the present invention, may be one in which one or more of the amino acid residues are substituted in a conserved or non-conserved manner, preferably a conserved manner in which one or more amino acid residues are substituted by residues of different structure but similar chemical characteristics, such as where a hydrophobic residues is substituted by a hydrophobic residue or where an acidic residue is substituted by another acidic residues or a polar residue for a polar residue or a basic residue for a basic residue. However, it is also in accordance with the present invention that more radical substitutions may prove advantageous and such substituted amino acid residue may even include one not encoded by the genetic code, or one in which one or more of the amino acid residues includes a substituent group not normally found among the amino acids in nature, at least without some type of in vivo modification.

In addition, the immunogens of the present invention may comprise, as part of, or attached to, the scaffold, or framework, an additional compound, or structure, such as a structure to increase the half-life of the polypeptide (for example, polyethylene glycol), or one in which the additional amino acids are fused to the mature polypeptide, such as a leader or secretory sequence or a sequence which is employed for purification of the mature polypeptide or a proprotein sequence. Such fragments, derivatives and analogs are deemed to be within the scope of those skilled in the art given the teachings herein.

The amino acid sequence disclosed in the Figures herein are in no way critical to the immunogens of the present invention but are merely specific embodiments of scaffolded, or frameworked, structures useful in practicing the present invention.

As used herein, the term “antigenic” refers to any biological structure, such as a polypeptide, or fragments thereof, but not limited thereto, that exhibits the ability to bind to an antibody, in vitro or in vivo, but which may or may not elicit the production of antibodies in response to administration of said antigenic structure to an animal. The term “immunogenic” refers to such a biological structure that, when administered to an animal, such as by intravenous or intramuscular injection, elicits the production in said animal of antibodies specific for the biological structure so administered. Use of these terms is well known to those skilled in the immunological and vaccine technological arts and will not be further discussed herein.

The polypeptides forming the immunogens of the present invention can be readily prepared by synthesis of, or by direct cloning of, polynucleotides encoding these polypeptides, or fragments thereof. Methods for doing so are well known in the art and will not be elaborated on further herein except to refer to certain references that find use in such preparations. See: Sambrook, et al., Molecular Cloning: A Laboratory Manual, Second Edition, Cold Spring Harbor, N.Y., (1989); Wu et al, Methods in Gene Biotechnology (CRC Press, New York, N.Y., 1997); and Recombinant Gene Expression Protocols, in Methods in Molecular Biology, Vol. 62, (Tuan, ed., Humana Press, Totowa, N.J., 1997), the disclosures of which are hereby incorporated by reference in their entirety.

In carrying out the procedures of the present invention it is of course to be understood that reference to particular buffers, media, reagents, cells, culture conditions and the like are not intended to be limiting, but are to be read so as to include all related materials that one of ordinary skill in the art would recognize as being of interest or value in the particular context in which that discussion is presented. For example, it is often possible to substitute one buffer system or culture medium for another and still achieve similar, if not identical, results. Those of skill in the art will have sufficient knowledge of such systems and methodologies so as to be able, without undue experimentation, to make such substitutions as will optimally serve their purposes in using the methods and procedures disclosed herein.

In accordance with the present invention, it must be appreciated that the design of a structural framework for antigen presentation should consist of a framework or scaffold having a number of desirable and advantageous characteristics that facilitate the attainment of the desired immunogenic structures. These characteristics include, but are not necessarily limited to, a small framework, minimal inherent antigenicity, conformational stability, and a structure that is known.

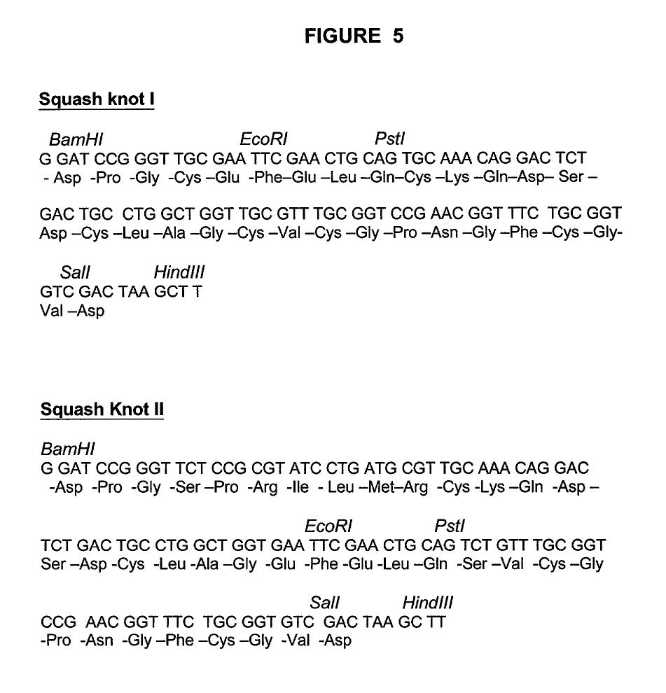

As a particular but non-limiting embodiment of the present invention, a small disulfide-rich protein from squash, designated “EETI-2” and which has a trypsin inhibitory function has been described and characterized by two-dimensional NMR techniques. [See Christmann et al, The Cysteine Knot of a squash-type protease inhibitor as a structural scaffold for Escherichia coli cell surface display of structurally constrained peptides, Protein Engineering, 12, 797-806 (1999); Heitz et al, Min-21 and Min-23, the smallest peptides that fold like a cysteine-stabilized β-sheet motif: design, solution structure, and thermal stability, Biochemistry, 38, 10615-10625 (1999)] (See FIG. 5)

The EETI-2 protein folds into a conformation that has been termed a “cysteine-stabilized β-sheet motif” (CSB) and has 3 β-strands bounded by loops and an α-helix (see the figures in the Christmann et al (1999) reference, the disclosure of which is incorporated herein by reference in its entirety).

In accordance with the latter embodiment, peptide antigens, or epitopic-peptides, derived from RSV F protein, such as from the A/II site, or other RSV antigen, are inserted into the a loop present in the EETI-2 structure (SEQ ID NO: 1 and in FIG. 1), where the disulfide linkages are indicated. Thus, in accordance with the present invention, the insertion sites are in the loop regions of the protein scaffold and the latter structure effectively tethers the ends of the peptide antigens, thereby stabilizing the polypeptide insert required for presentation of the proper antigenic conformation.

In a specific embodiment of the present invention, the immunogens, including immunogenic recombinant oligopeptides or polypeptides, of the present invention are inserted in place of residues 3-8 (from the proline to the arginine) in the sequence of SEQ ID NO: 1, thereby replacing the proline to arginine segment of this portion of the EETI-2 protein. Thus, to produce the immunogens, or immunogenic recombinant polypeptides, of the present invention this hexapeptide sequence of EETI-2 (SEQ ID NO: 1) is replaced by a peptide-epitope derived from RSV F protein, which may involve a sequence of at least about 20 to at most about 100 residues. The exact size of such a replacement sequence depends on the size of the fragment derived from F protein, or other RSV antigen, that is sufficient to confer the desired immunogenic activity on the resulting protein structure. In addition, peptide linker sequences can also be inserted at the ends of the immunogenic polypeptides so as to bind them to the β-sheet structures of EETI-2 and thereby hold the immunogenic recombinant polypeptides in place.

While the native EETI-2 structure may itself permit 3 loops to be held together by its β-sheet structures, in accordance with the present invention no such limitation need be accepted and, using principles of genetic engineering, as well known to those skilled in the art, immunogens, including immunogenic recombinant polypeptides, of the present invention may be produced that contain more than three looping regions and thus more than three immunogenic structures for presentation to the immune system of the animal to be vaccinated with the immunogenic compositions disclosed herein.

Structural studies have shown that the EETI-2 protein, with a deletion at the amino terminus, is not adversely effected as to overall conformation and thus the disulfide bond between the cysteines at residues 2 and 19 is not critical for proper folding (although loss of this disulfide bond causes a slight decrease in thermal stability to about 100° C. [See: Heitz et al (1999), the disclosure of which is hereby incorporated by reference in its entirety]. Deleting of this disulfide bond thereby is expected to produce increased flexibility of the protein structure in the region of the loop at amino acid residue number 18. [See: FIG. 2 and sequence therein] Thus, this modified form of the EETI-2 protein, with the ability to accept insertions at amino acid 18 (and altering the cysteines at positions 2 and 19 into serines) may allow insertions of peptide antigens of large size and thereby readily accommodates the polypeptide inserts of the present invention. [See: FIG. 3 and sequence therein].

The present invention also relates to immunogenic compositions, such as vaccine compositions, comprising the immunogens and immunogenic compositions disclosed herein. Such an immunogenic composition is a composition comprising at least one of the immunogens disclosed herein wherein said immunogen is suspended in a pharmacologically acceptable carrier.

The present invention further relates to a vaccine composition comprising the immunogenic compositions disclosed herein.

Generally, vaccines are prepared as injectables, in the form of aqueous solutions or suspensions. Vaccines in an oil base are also well known such as for inhaling. Solid forms which are dissolved or suspended prior to use may also be formulated. Pharmaceutically acceptable carriers, diluents and excipients are generally added that are compatible with the active ingredients and acceptable for pharmaceutical use.

The pharmaceutical compositions useful herein also contain a pharmaceutically acceptable carrier, including any suitable diluent or excipient, which includes any pharmaceutical agent that does not itself induce the production of antibodies harmful to the individual receiving the composition, and which may be administered without undue toxicity. Pharmaceutically acceptable carriers include, but are not limited to, liquids such as water, saline, glycerol and ethanol, and the like, including carriers useful in forming sprays for nasal and other respiratory tract delivery or for delivery to the ophthalmic system. A thorough discussion of pharmaceutically acceptable carriers, diluents, and other excipients is presented in REMINGTON'S PHARMACEUTICAL SCIENCES (Mack Pub. Co., N.J. current edition).

Vaccine compositions may further incorporate additional substances to stabilize pH, or to function as adjuvants, wetting agents, or emulsifying agents, which can serve to improve the effectiveness of the vaccine.

Vaccines are commonly administered along with an adjuvant in order to potentiate the immunological effects of the vaccine. In the present invention, such adjuvants may also accompany the epitope-based vaccines disclosed herein but, in addition, such epitopes may also serve in the capacity of adjuvants for administration with other vaccines. For example, in providing a vaccine, such as a whole protein vaccine or an attenuated vaccine formed from the organism itself, such as a heat or chemically attenuated virus, the epitopic structures of the present invention, such as the immunogens disclosed herein, may likewise be administered along with other immunogenic structures in order to enhance the immunological effects of such compositions.

Vaccines are generally formulated for parenteral administration and are injected either subcutaneously or intramuscularly. Such vaccines can also be formulated as suppositories or for oral administration, using methods known in the art, or for administration through nasal or respiratory routes.

The amount of vaccine sufficient to confer immunity to pathogenic organisms, such as viruses, especially RSV, or other microbes is determined by methods well known to those skilled in the art. This quantity will be determined based upon the characteristics of the vaccine recipient and the level of immunity required. Where vaccines are administered by subcutaneous or intramuscular injection, a range of 0.5 to 500 μg purified protein may be given. As useful in the present invention, such dosages are commonly sufficient to provide about 1 μg, possibly 10 μg, even 50 μg, and as much as 100 μg, up to 500 μg of immunogenic protein, or immunogenic polypeptide, or immunogenically active fragments thereof. In addition, more than one such active material may be present in the vaccine. Thus, more than one antigenic structure may be used in formulating the vaccine, or vaccine composition to use in the methods disclosed herein. This may include two or more individually immunogenic proteins or polypeptides, proteins or polypeptides showing immunogenic activity only when in combination, either quantitatively equal in their respective concentrations or formulated to be present in some ratio, either definite or indefinite. Thus, a vaccine composition for use in the processes disclosed herein may include one or more immunogenic proteins, one or more immunogenic polypeptides, and/or one or more immunogenically active immunogens comprising antigenic fragments of said immunogenic proteins and polypeptides, the lafter fragments being present in any proportions selected by the use of the present invention.

The present invention is also directed to a process for preventing or treating a disease comprising administering to a patient having said disease, or at risk of contracting said disease, a therapeutically, or prophylactically, effective amount of the vaccine compositions of the present invention. In such treatment or prevention, the disease is commonly a disease of the respiratory system, especially where said disease is caused by a virus, most especially where said virus is RSV.

The present invention will now be further described by way of the following non-limiting example. In applying the disclosure of the example, it should be kept clearly in mind that other and different embodiments of the methods disclosed according to the present invention will no doubt suggest themselves to those of skill in the relevant art.

### EXAMPLE 1

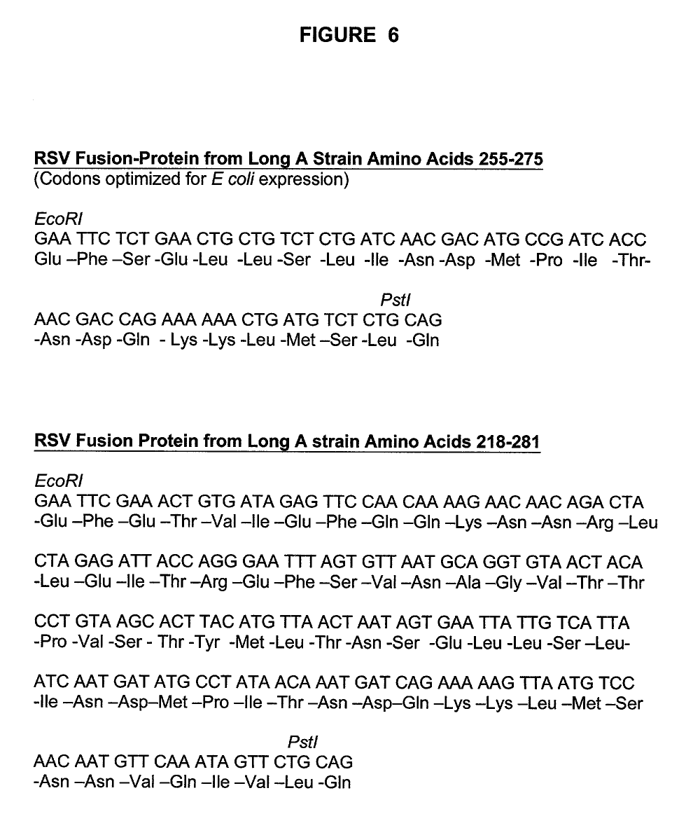

An EETI-2 with an insertion site at position 3 (SK1) or an EETI-2 with an insertion site at position 18 (SK2) were made to have insertions of 0, 20 or 63 amino acid insertions from the RSV F-protein from Strain A Long. The 20 amino acid insert is comprised of amino acids 255-275 and the 63 amino acid insert is comprised of amino acids 218-281 of the RSV F-protein. (See FIG. 6)

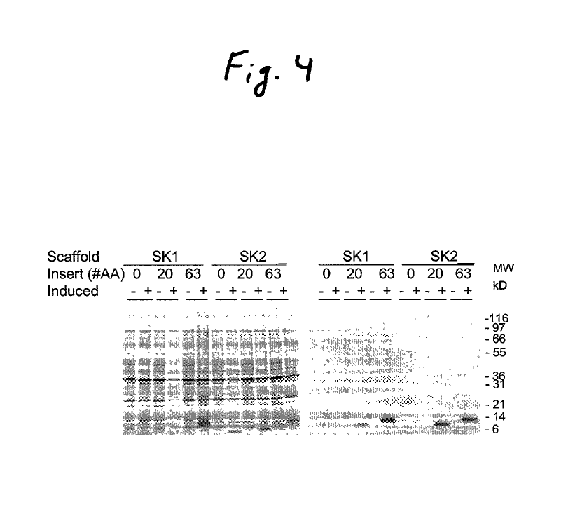

These constructs were put into a T7 expression plasmid (pMSH26) containing a Braun's lipoprotein signal sequence in frame with the inserted gene. These constructs produce a protein which is targeted to the membrane via the lipidation of the amino terminal cysteine and will be folded in the periplasmic space. Induction of the T7 promotor by addition of IPTG (isopropylthiogalactoside) causes an overproduction of the protein that can be visualized by probing with MEDI-493 on a western blot format (See FIG. 4). The protein is found in a sediment of disrupted F protein epitope-expressing cells resulting from high speed centrifugation corresponds to the membrane fraction of the E. coli cell.

What is claimed is:

1. An immunogen for eliciting neutralizing antibodies against respiratory syncytial virus (RSV), said immunogen comprising a non-RSV polypeptide fragment fused to a neutralizing epitope derived from the F protein of RSV, said immunogen being free of immunodominant non-neutralizing RSV epitopes.

2. The immunogen of claim 1 wherein said immunogen has been depleted of immunodominant non-neutralizing RSV epitopes.

3. The immunogen of claim 1 wherein said immunogen is a single chain polypeptide.

4. The immunogen of claim 1 wherein said immunogen comprises more than one polypeptide.

5. The immunogen of claim 1 wherein said neutralizing epitope is derived from the A/II region of RSV F protein.

6. The immunogen of claim 1 wherein said neutralizing epitope of RSV is the amino acid sequence of the A/II F region of protein of RSV.

7. The immunogen of claim 1 wherein said conformational epitope specifically binds to an antibody specific for the F antigen of RSV.

8. The immunogen of claim 7 wherein said antibody is MEDI-493.

9. The immunogen of claim 7 is an antibody with the same specificity as MEDI-493.

10. The immunogen of claim 9 wherein said antibody has at least the same affinity for the conformational epitope as does MEDI-493.

11. The immunogen of claim 1 wherein said non-RSV fragment and said polypeptide insert derived from the RSV F protein are joined to each other by at least two peptide bonds.

12. The immunogen of claim 1 wherein said non-RSV fragment is derived from EETI-2 protein

13. The immunogen of claim 12 wherein said non-RSV fragment is EETI-2 protein

14. An immunogenic composition comprising at least one of the immunogens selected from the group consisting of the immunogens of claims 1, 2, 3, 4, 5, 6, 7, 8, 9, 10, 11, 12, and 13 and wherein said immunogen is suspended in a pharmacologically acceptable carrier.

15. A vaccine composition comprising the immunogenic composition of claim 14.

16. A process of preventing or treating a disease comprising administering to a patient having said disease, or at risk of contracting said disease, a therapeutically, or prophylactically, effective amount of the vaccine composition of claim 15.

17. The process of claim 15 wherein said disease is a disease of the respiratory system.

18. The process of claim 17 wherein said disease is caused by a virus.

19. The process of claim 18 wherein said virus is RSV.


SEQUENCE LISTING (9 sequences)

SEQ ID NO: 1 - protein, 28 aa, Artificial Sequence
  Description of Artificial Sequence EETI-2 protein from squash.
GCPRILMRCKQDSDCLAGCVCGPNGFCG

SEQ ID NO: 2 - DNA, 101 bp, Artificial Sequence
  Description of Artificial Sequence Squash Knot I nucleotide sequence.
ggatccgggttgcgaattcgaactgcagtgcaaacaggactctgactgcctggctggttgcgtttgcggtccgaacggtttctgcggtgtcgactaagctt

SEQ ID NO: 3 - protein, 31 aa, Artificial Sequence
  Description of Artificial Sequence Squash Knot I protein.
DPGCEFELQCKQDSDCLAGCVCGPNGFCGVD

SEQ ID NO: 4 - DNA, 119 bp, Artificial Sequence
  Description of Artificial Sequence Squash Knot II nucleotide sequence.
ggatccgggttctccgcgtatcctgatgcgttgcaaacaggactctgactgcctggctggtgaattcgaactgcagtctgtttgcggtccgaacggtttctgcggtgtcgactaagctt

SEQ ID NO: 5 - protein, 37 aa, Artificial Sequence
  Description of Artificial Sequence Squash Knot II protein.
DPGSPRILMRCKQDSDCLAGEFELQSVCGPNGFCGVD

SEQ ID NO: 6 - protein, 25 aa, Artificial Sequence
  Description of Artificial Sequence RSV Fusion Protein.
EFSELLSLINDMPITNDQKKLMSLQ

SEQ ID NO: 7 - DNA, 75 bp, Artificial Sequence
  Description of Artificial Sequence RSV Fusion Protein nucleotide sequence.
gaattctctgaactgctgtctctgatcaacgacatgccgatcaccaacgaccagaaaaaactgatgtctctgcag

SEQ ID NO: 8 - protein, 68 aa, Artificial Sequence
  Description of Artificial Sequence RSV Fusion Protein.
EFETVIEFQQKNNRLLEITREFSVNAGVTTPVSTYMLTNSELLSLINDMPITNDQKKLMSNNVQIVLQ

SEQ ID NO: 9 - DNA, 204 bp, Artificial Sequence
  Description of Artificial Sequence RSV Fusion Protein nucleotide sequence.
gaattcgaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttctgcag